(19) United States
(12) Patent Application Publication
(10) Pub. No.: US 2014/0235963 A1
(43) Pub. Date: Aug. 21, 2014

(54) Remote Health Care System

(71) Applicant: Welch Allyn, Inc., Skaneateles Falls, NY (US)

(72) Inventors: Ian K. Edwards, Teton Village, NY (US); Raymond A. Lia, Auburn, NY (US); David J. Maier, Skaneateles, NY (US); Miguel Christopher Mudge, Skaneateles, NY (US); Jon R. Salvati, Auburn, NY (US); Robert L. Vivenzio, Auburn, NY (US); Robert J. Wood, Marco Island, FL (US)

(73) Assignee: Welch Allyn, Inc., Skaneateles Falls, NY (US)

(21) Appl. No.: 14/180,380

(22) Filed: Feb. 14, 2014

Related U.S. Application Data
(60) Provisional application No. 61/765,341, filed on Feb. 15, 2013.

(51) Int. Cl. A61B 5/00; A61B 5/0205; A61B 5/11; A61B 5/145

(52) U.S. Cl. CPC A61B 5/0022; A61B 5/14542; A61B 5/14532; A61B 5/02055; A61B 5/1117; A61B 5/0077; A61B 5/7405; A61B 5/742
USPC 600/301; 600/300; 600/485; 600/364; 600/365; 600/549; 600/499

(57) ABSTRACT

A health care medical device includes one or more medical sensor devices, an assembly that houses the one or more medical sensor devices and a mobile electronic computing device with a display. The mobile electronic computing device is secured to the assembly. Medical data from the one or more medical sensor devices is transmitted to the mobile electronic computing device.

### BACKGROUND

Health care monitoring has become an important part of the practice of medicine. When patients have been diagnosed with a generally treatable condition such as high blood pressure, diabetes and certain breathing disorders, health care professionals often request that patients monitor their conditions in their home. Providing health records of such medical parameters as blood pressure, glucose levels and oxygen saturation can be an important aid to helping help care professionals treat their patients.

Medical devices like blood pressure monitors, glucose monitors and oxygen saturation monitors are commonly available for a remote environment. These medical devices are typically available as separate units. Many of these medical devices may be difficult to use, particularly for patients that are visually impaired, mechanically challenged or generally averse to technology.

### SUMMARY

Embodiments of the disclosure are directed to a health care medical device. The health care medical device comprises one or more medical sensor devices, an assembly that houses the one or more medical sensor devices and a mobile electronic computing device with a display. The mobile electronic computing device is secured to the assembly and medical data from the one or more medical sensor devices is transmitted to the mobile electronic computing device.

In another aspect, a method of monitoring medical parameters of a patient using a health care medical device comprises placing one or more medical sensors from the health care medical device on the patient. Data is received from the one or more medical sensors. The received data is processed into medical parameters associated with the one or more medical sensors. Medical data based on the medical parameters is sent parameters to a mobile electronic computing device that is part of the health care medical device. The medical data is displayed on a display screen of the mobile electronic computing device. A wireless connection is made from the mobile electronic computing device to an Internet-based storage system. The medical data is sent to the Internet-based storage system.

In yet another aspect, a health care medical device comprises one or more medical sensor devices, a tablet computer and an assembly that houses the one or more medical sensor devices and the tablet computer. The medical sensor devices include one or more of a blood pressure sensor, oxygen saturation sensor, thermometer and glucose meter. The assembly encloses the back and at least a part of four sides of the tablet computer. The tablet computer provides visual and oral prompts to a user during operation of the health care medical device and the tablet computer transmits medical data from the one or more medical sensor devices to an Internet-based storage system.

The details of one or more techniques are set forth in the accompanying drawings and the description below. Other features, objects, and advantages of these techniques will be apparent from the description, drawings, and claims.

### DESCRIPTION OF THE DRAWINGS

FIG. 1 shows an example system that supports a remote health care system.

FIG. 2 shows an example drawing of the remote health care medical device of FIG. 1.

FIG. 3 shows example modules of the remote health care medical device of FIG. 2.

FIG. 4 shows example modules of the base assembly of the remote health care medical device of FIG. 2.

FIG. 5 shows an example start screen for the remote health care medical device of FIG. 2.

FIG. 6 shows an example introduction screen for a temperature measurement taken using the remote health care medical device of FIG. 2.

FIG. 7 shows an example instruction screen for the temperature measurement.

FIG. 8 shows an example result screen for the temperature measurement.

FIG. 9 shows an example introduction screen for an oxygen saturation measurement taken using the remote health care medical device of FIG. 2.

FIG. 10 show an example instruction screen for the oxygen saturation measurement.

FIG. 11 shows an example result screen for the oxygen saturation measurement.

FIG. 12 shows an example introduction screen for a glucose measurement taken using the remote health care medical device of FIG. 2.

FIG. 13 shows an example instruction screen for the glucose measurement.

FIG. 14 shows an example result screen for the glucose measurement.

FIG. 15 shows an example introduction screen for a blood pressure measurement taken using the remote health care medical device of FIG. 2.

FIG. 16 shows an example instruction screen for the blood pressure measurement.

FIG. 17 shows another example instruction screen for the blood pressure measurement.

FIG. 18 shows an example result screen for the blood pressure reading.

FIG. 19 shows a flowchart for a method for obtaining medical data from a sensor using the remote health care medical device of FIG. 2.

FIG. 20 shows example physical components of the remote health care medical device of FIG. 2.

### DETAILED DESCRIPTION

The present disclosure is directed to a health care system that integrates a plurality of medical sensor devices in a single unit. The health care system is designed so that it is easy to use. In some examples, visual and oral prompts are included to provide instructions and reminders to patients. A compute engine such as a tablet computer or smart telephone controls the visual and oral prompts, receives and displays medical data from the medical sensor devices and transmits the medical data to an Internet-based storage system.

In one example, the health care system includes a base unit and the compute engine. The base unit includes medical sensor devices such as a blood pressure monitor, thermometer, glucose monitor and oxygen saturation sensor. The compute engine interfaces with the base unit and with the Internet-based storage system. The Internet-based storage system may be a web service, an Internet-based cloud service or an Internet-exposed database. Once the medical data is stored in the Internet-based storage system, the medical data may be sent to the patient, an electronics medical records (EMR) system, to a physician's office, and/or to any other appropriate location for analysis of the medical data.

The health care system described in this disclosure is designed to be used in a location remote from a health care provider. For example, the health care system may be used in a home environment. In other examples, the health care system may be used in applications outside the home, for example in a clinic or in a commercial setting such as a supermarket, retail store or similar setting. In this disclosure, the health care system may be referred to as a remote health care system. In addition, in this disclosure, a health care medical device may be referred to as a remote health care medical device.

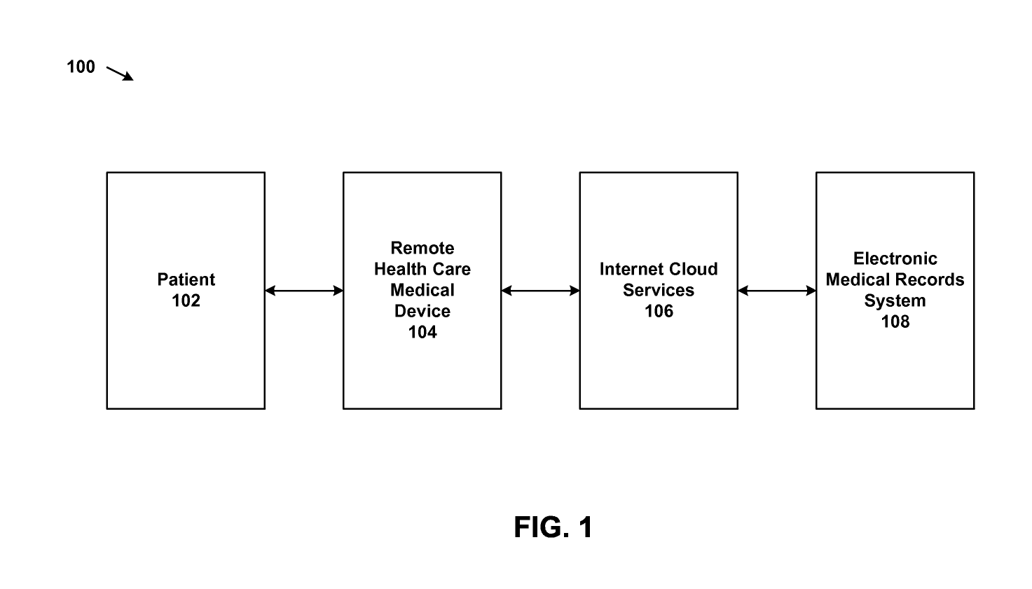

FIG. 1 shows an example system 100 that supports a remote health care system. The example system 100 includes a patient 102, a remote health care medical device 104, an Internet cloud service 106 and an EMR system 108. More, fewer, or different components of the example system 100 may be used.

The example remote health care medical device 104 is a self-contained unit that includes a plurality of medical sensor devices and the compute engine. In some examples, the medical sensor devices include a blood pressure monitoring device, a thermometer, a glucometer and an oxygen saturation sensor. More, fewer or different medical sensor devices may be used. The compute engine is a mobile electronic computing device with a display, such as a tablet computer or a smart telephone.

The medical sensor devices are attached or connected to patient 102. Medical data from the sensor devices is sent to the compute engine for display and for transmission to an Internet-based storage system. In the example system 100, the Internet-based storage system is a third party source, such as Internet cloud services 106. Ease of use of the remote health care medical device 104 is facilitated via instructions, both oral and visual from the compute engine. Ease of use is also facilitated via using the compute engine to transmit the medical information to the Internet cloud services 106.

The example Internet cloud services 106 is typically a third party Internet services provider, such as Amazon web services. In the example system 100, a health care website is hosted on Internet cloud services 106. A health care application may be downloaded from the health care website and installed on the compute engine of the remote health care medical device 104. Medical sensor data received at the remote health care medical device 104 may be transmitted to the health care website. The health care website is typically configured to send the medical sensor data to a final destination—for example the EMR system 108, a physician's office, etc. The example health care website running on Internet cloud services 106 may receive medical sensor data from a plurality of remote health care medical devices.

The example EMR system 108 is an electronic medical records system for storing medical data for patients. Example information stored in the EMR system 108 includes identification information for the patient (name, address, patient identification number) and a medical history for the patient including records of blood pressure, temperature, glucose levels, oxygen saturation and other vital sign data for the patient.

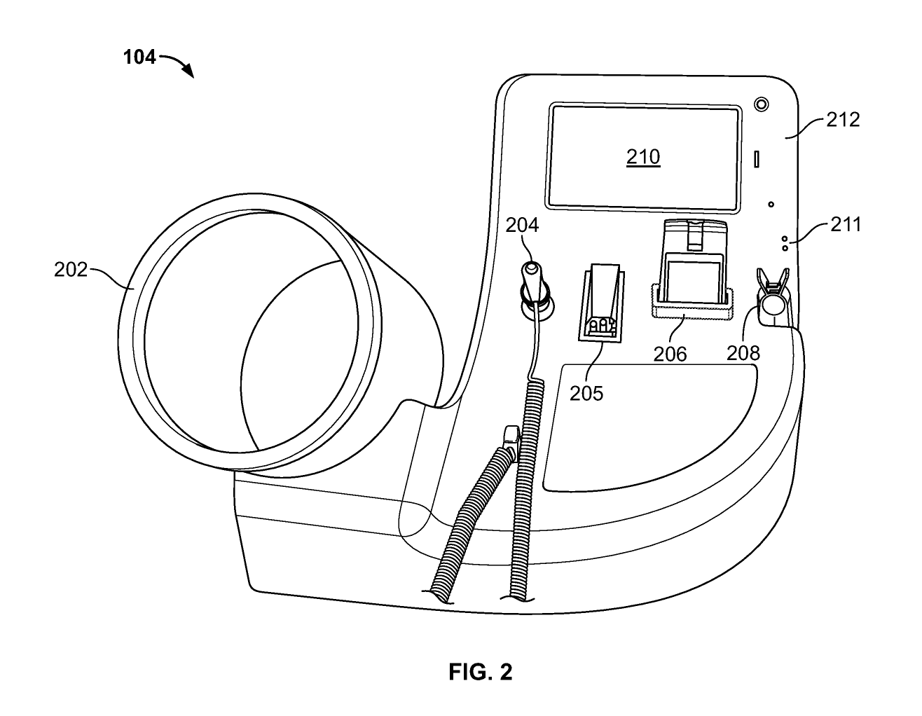

FIG. 2 shows an example drawing of the remote health care medical device 104. The remote health care medical device 104 includes a blood pressure sleeve 202, a thermometer probe 204, covers for the thermometer probe 205, a glucometer 206, an oxygen saturation sensor 208, a tablet computer 210 and speaker holes 211.

The blood pressure sleeve 202 is used to take a patient's blood pressure. The patient inserts an arm into the blood pressure sleeve 202. When a blood pressure reading is started, a blood pressure cuff inflates in the blood pressure sleeve 202. After reaching a suitable inflation, the cuff deflates and the patient's blood pressure is measured. In some examples, the blood pressure cuff automatically adjusts in position around the patient's arm to properly read the patient's blood pressure. The automatic adjustment of the blood pressure cuff ensures that blood pressure cuff is in a proper position to read the patient's blood pressure. The blood pressure sleeve 202, in conjunction with the automatic adjustment of the blood pressure cuff, also obviates the need of a patient to manually strap, secure and position the blood pressure cuff around the patient's arm.

The thermometer probe 204 and the oxygen saturation sensor 208 are typically hard-wired to the console of the remote health care medical device 104, although wireless sensors are possible. The glucometer 206 is typically a portable device. Typically, a user takes a glucose strip and puts the glucose strip into the glucometer 206. The user lances a finger and applies blood to the glucose strip. When the blood is applied to the glucose strip, the glucometer displays a glucose reading for the patient.

The tablet computer 210 comprises the compute engine for the remote health care medical device 104. As shown in FIG. 2, the tablet computer 210 is embedded in enclosure 212. Embedding the tablet computer 210 in the enclosure 212 secures the tablet computer 210 in the enclosure 212 and provides the appearance of a seamless health care medical device unit. Embedding the tablet computer 210 to provide a seamless health care medical device unit may make the remote health care medical device 104 appear more user friendly to older or technology adverse people. As shown in FIG. 2, the back, and four sides of the tablet computer 210 are embedded in enclosure 212, leaving the display screen of the tablet computer 210 available for viewing.

In other examples, instead of being embedded in enclosure 212, the tablet computer 210 may be secured to enclosure 212 with a mounting bracket. A plurality of mounting brackets may be designed. For example, the enclosure 212 may include a mounting bracket designed to hold and secure tablet computer 210. In other examples, the enclosure 212 may include a mounting bracket designed to hold and secure a smart telephone. For example, in one alternative, the enclosure 212 can simply be a cradle that holds a smartphone or other similar device and allows the device to be connected to the remote health care medical device 104 using a wired connection to a USB or similar port, or using a wireless connection such as Bluetooth or Near Field Communication (NFC). Other types of mechanical assemblies or wireless connections may be used.

The enclosure 212 also includes example speaker holes 211. The speaker holes 211 provide openings so that audio from the tablet computer 110 may be heard. The audio is typically used for oral prompting and instructions during operation of the remote health care medical device 104. Oral prompting is explained later herein. In other examples, the audio may originate from the smart telephone or other similar device.

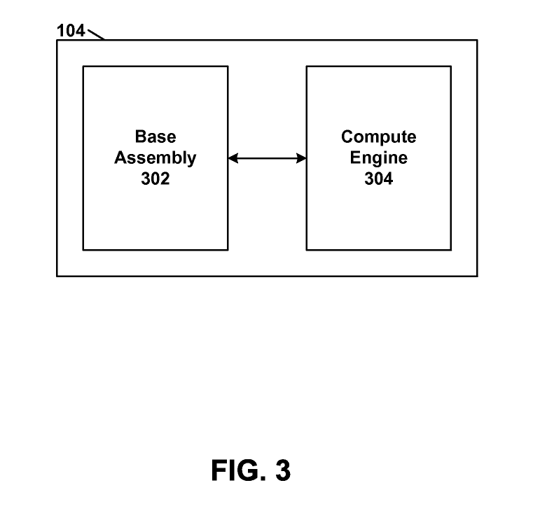

FIG. 3 shows example modules of the remote health care medical device 104. The remote health care medical device 104 includes base assembly 302 and compute engine 304. The example base assembly 302 is an assembly that includes the medical sensor devices of the remote health care medical device 104. Example medical sensor devices included in the base assembly 302 include a blood pressure sensor, thermometer, glucometer and oxygen saturation sensor. The compute engine 304 is typically a mobile electronic device, such as a tablet computer or a smart telephone. In some examples, the compute engine 304 is wirelessly connected to the base assembly 302 via a wireless communications mechanism such as Bluetooth. In other examples, the compute engine may be physically connected to the base assembly 302 via a USB connector or by some other physical connection.

The compute engine 304 facilitates transmitting medical data from the medical sensor devices to a final destination, such as EMR system 108, a physician's office, etc. Because of the compute engine 304, a patient using the remote health care medical device 104 need not be concerned about transmitting the medical data to the final destination. The compute engine 304 transmits the medical data to Internet cloud services 106, and Internet cloud services 106, for example the health care website running on Internet cloud services 106, transmits the medical data to a configured destination, such as EMR system 108. Separating the base assembly 302 from the compute engine 304 also permits easy upgrade of the compute engine 304 when technology changes. For example, an upgrade simply consists of replacing the tablet computer or smart telephone with a newer model. Separating the base assembly 302 from the compute engine 304 also permits the base assembly 302 to be sold as a separate product, without the compute engine 304. For example, a customer may want to provide their own compute engine, such as their own tablet computer or smart telephone.

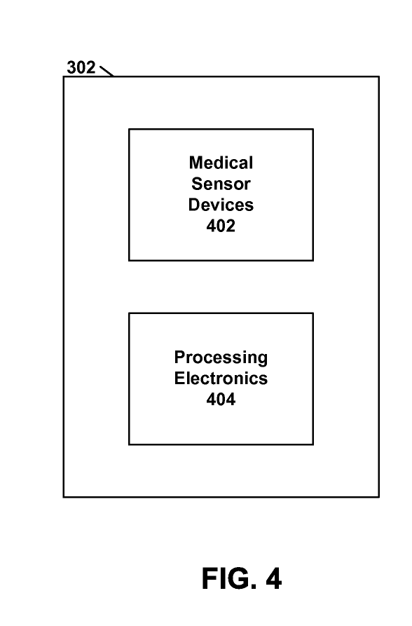

FIG. 4 shows example modules of the base assembly 302. The base assembly 302 includes medical sensor devices 402 and processing electronics 404. Raw medical data received from medical sensor devices 402 is translated into formatted medical data by processing electronics 404. For example, blood pressure data transmitted by a blood pressure monitor may comprise pulses of electrical data. The processing electronics 404 converts the blood pressure data into a blood pressure reading comprising a systolic blood pressure divided by a diastolic blood pressure. The blood pressure reading, comprising the systolic blood pressure divided by the diastolic blood pressure, is transmitted to the compute engine 304. In some examples, processing electronics for each medical sensor device is included on the medical sensor device. In other examples, the base assembly 302 may include common processing electronics 404 for all the medical sensor devices 402.

As discussed, medical sensor devices 402 may include medical sensor devices for monitoring blood pressure, temperature, glucose levels and oxygen saturation. Other medical sensor devices may be included in the remote health care medical device 104. For example, the remote health care medical device 104 may include a fall sensor that determines whether a person has fallen. In some examples, the fall sensor comprises an accelerometer that is attached to the patient near the patient's body frame. A wireless connection, for example Bluetooth, may connect the accelerometer to the compute engine 304, either tablet computer 210 or a smart telephone. In some examples, a software algorithm in the accelerometer places a variety of acceleration event magnitudes on a timeline. Events that occur in an order that resembles a patient fall cause a peak in the timeline that may be used to determine risk. A combination of events at different times can indicate that the patient has fallen.

Another medical sensor device that may be included in the remote health care medical device 104 is a weight sensor. In some examples, the weight sensor may be installed on a chair that may be part of the remote health care medical device 104. When the patient sits on the chair, the patient's weight is measured by the weight sensor. In some examples, the weight sensor may be a capacitive sensor. In some examples, the capacitive weight sensor comprises two flexible sheets with metal on both sides of the sheets. An insulator, such as a piece of rubber, may be included between the two flexible sheets. When the patient sits on the chair, the flexible sheets press against the rubber, resulting in a change of the capacitance of the weight sensor. Changes in capacitance can be correlated into changes of the patient's weight, and in this manner the patient's weight can be measured. Other types of weight sensors may be used.

In some examples, the remote health care medical device 104 may include a general purpose camera for viewing of the patient. Standard video technologies such as Skype or Face Time may be used to establish a connection with the camera. For example, the camera may be connected to a physician's office and used to examine the patient or to observe certain areas of the patient's body. The physician may be able to make a diagnosis using the camera. In some examples, the camera may be used by a clinician, for example a nurse, to verify that the patient has taken prescribed medication. For example, the patient may use the camera to show the clinician a pill box with the prescribed medication. The clinician may verify proper medication by the size, shape and color of the medication and also verify that the patient has taken the proper medication for a particular day. Other uses for the camera are possible.

In some examples, the remote health care medical device 104 may include additional user interfaces. For example, the tablet computer 210 may provide a manual input, for example via a touch screen, wherein the patient may answer a series of questions about what they are doing at the time they are using the remote health care medical device 104 and answer other questions such as medications taken, foods eaten, etc. In addition, alerts, such as an audible alarm or a telephone call, may prompt the patient to take readings on the remote health care medical device 104. Further, a doctor may be alerted if a patient is non-compliant. There may also be an early warning notification system, via messages on the tablet computer 210 or smart telephone when measured readings exceed a predetermined threshold. Alerts may also be provided when a determination is made that one or more medical sensor devices are producing invalid readings.

Other features of the remote health care medical device 104 may include transmitting medical sensor data to an artificial intelligence tool made available by Internet cloud services 106. For example, the artificial intelligence tool may determine trends from the medical data and may provide the trends to a physician for review.

Another feature may be to configure the Internet cloud services 106 to format information received from the remote health care medical device 104 in different ways and to direct the information to different parties. For example, Internet cloud services 106 may send all medical sensor data received from the remote health care medical device 104 to a physician and to EMR system 108. However, Internet cloud services 106 may direct a subset of the medical sensor data to an insurance company and Internet cloud services 106 may add billing codes to the medical sensor data before sending the medical sensor data to the insurance company.

As an additional use of Internet cloud services 106, a patient's medical records may be stored on Internet cloud services 106. Any medical sensor data sent to Internet cloud services 106 for the patient may be stored in the patient's record on the Internet cloud services 106. Once the patient's medical record is stored on Internet cloud services 106, a copy of the record may be made available to authorized medical personal, for example in an emergency situation. This concept may be similar to a patient wearing a medical alert bracelet or a dog tag that contains emergency medical information for the patient. An example of an Internet based-third party solution for storing a patient's health data is Microsoft Health Vault.

A further feature may be to adapt Internet cloud services 106 so that the medical sensor data may be directed to a large number of EMR systems. In practice, EMR systems may have different interfaces. By using a standard formatting technique such as XSLT (the extensible style sheet language transformation), the medical sensor data may be compatible with a larger number of EMR systems.

An additional feature may be the use of smart alerts. The remote health care medical device 104 may be programmed to perform periodic self-tests of medical sensor devices. When a determination is made that a medical sensor device is not working properly, instead of displaying a cryptic error message on the tablet computer 210, the remote health care medical device 104 may display a more user friendly message on the tablet computer 210. In addition, a more technical based error message may be sent to Internet cloud services 106 for transmission to maintenance personnel.

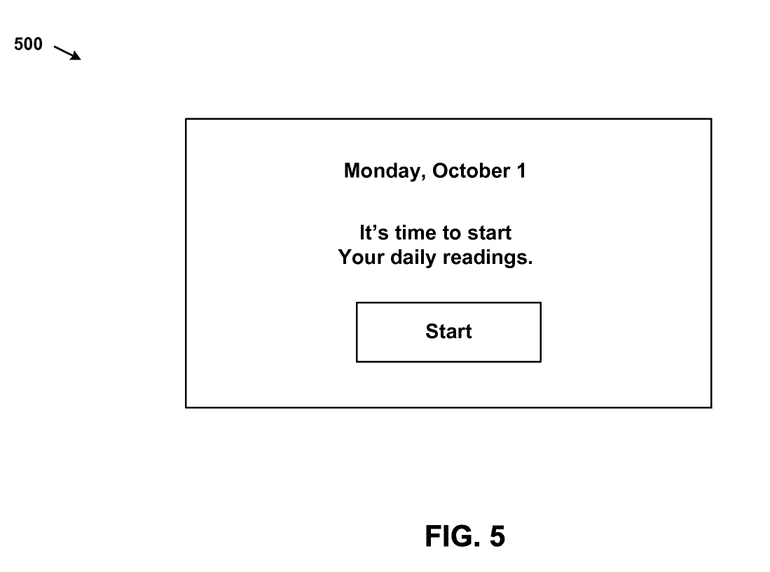

FIGS. 5-18 show example visual prompt screens that may appear on the tablet computer 210 during operation of the remote health care medical device 104. More, fewer, or different screens may be used. In addition, the screens may appear on a smart telephone instead of on tablet computer 210. In some examples, oral prompting may be used together with or in lieu of visual prompting.

FIG. 5 shows an example start screen 500. The start screen 500 may be configured to be displayed at a certain time of the day. The time of day may be configured as a time of day in which a patient wishes to use the remote health care medical device 104. The start screen 500 indicates the day and date. The start screen also displays a message, in this case “It's time to start your daily readings.” The start screen 500 also includes a Start button.

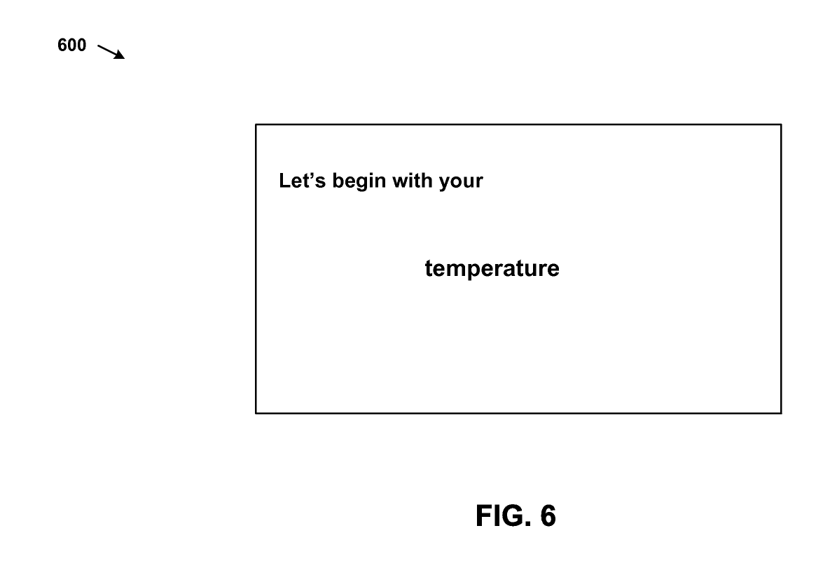

After the Start button is pressed, screen 600, shown in FIG. 6, is displayed. Screen 600 displays a message indicating that temperature is the first measurement to be obtained.

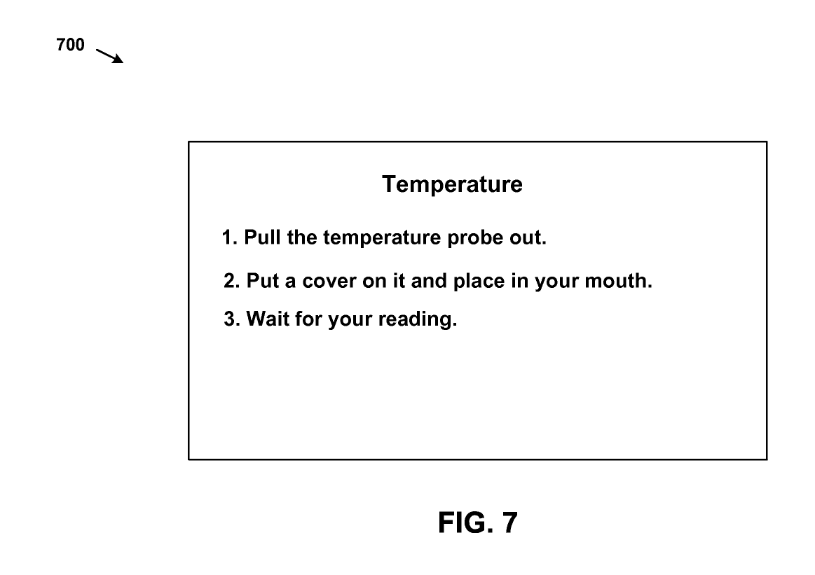

After an appropriate time for the user to view screen 600, for example after a few seconds, screen 700, shown in FIG. 7 is displayed. Screen 700 shows example instructions for obtaining a temperature reading. The example instructions include 1) pull the temperature probe out, 2) put a cover on the temperature probe and place the covered temperature probe in your mouth, and 3) wait for your reading.

In example embodiments, the transitions between each screen can be manually-actuated or automatic. For example, a button or other similar prompt can be provided to allow the patient to manually move through each screen. In another example, the system can be configured to automatically move between screens based upon elapsed time or sensors. For example, the system can sense when a blood pressure cuff is removed from the device and provide instructions upon the removal. Other configurations are possible.

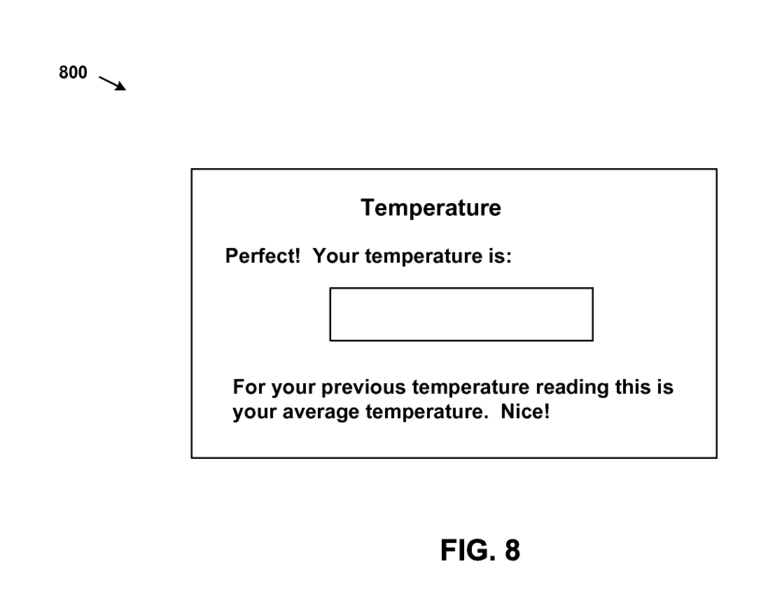

FIG. 8 shows an example result screen 800 for temperature. The result screen 800 shows the patient's temperature reading and displays a message providing feedback for the patient. For example, the message displayed in result screen 800 indicates that the temperature reading is consistent with previous readings. If the temperature reading were too high, the message could indicate an action for the patient to take, for example to call a doctor.

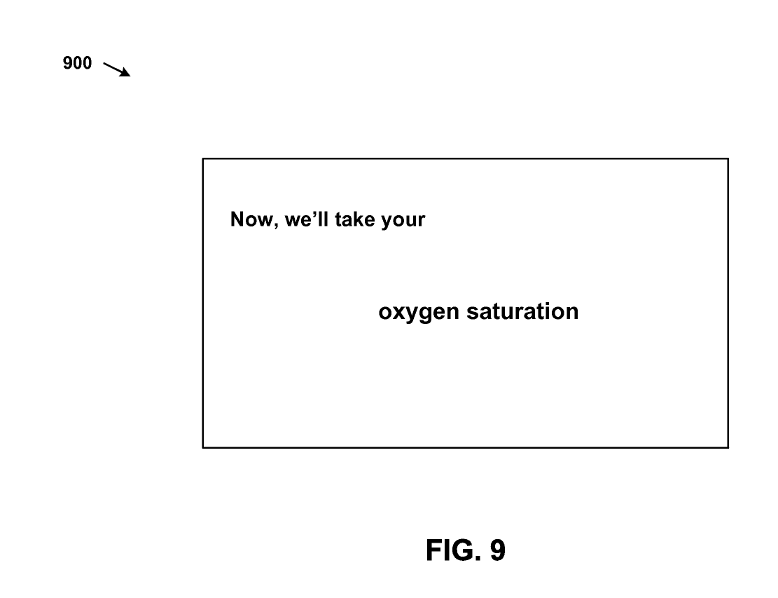

FIG. 9 shows an example screen 900 that may be displayed after the result screen 800. The screen 900 indicates to the patient that the patient's oxygen saturation is to be taken next.

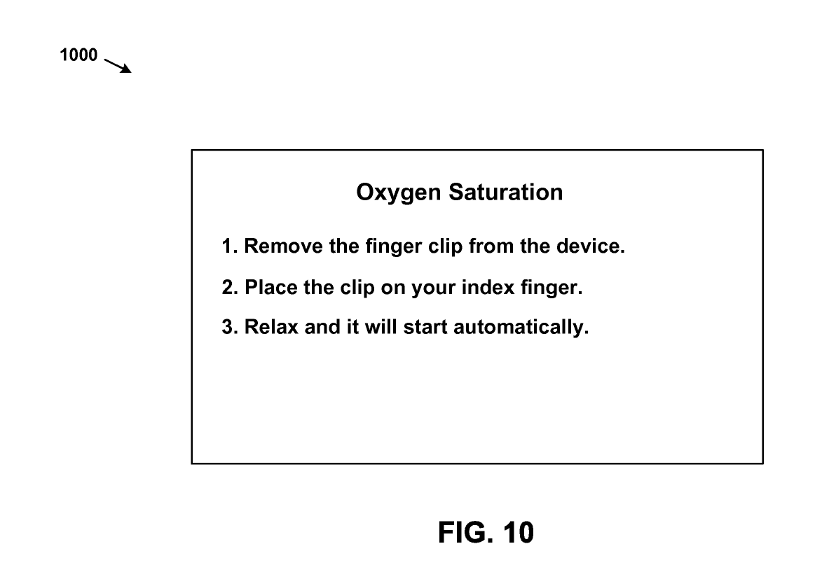

FIG. 10 shows an example instruction screen 900 for oxygen saturation. The example instructions include 1) remove the finger clip from the device; 2) place the clip on your index finger; and 3) relax and it will start automatically. Other instructions may be used.

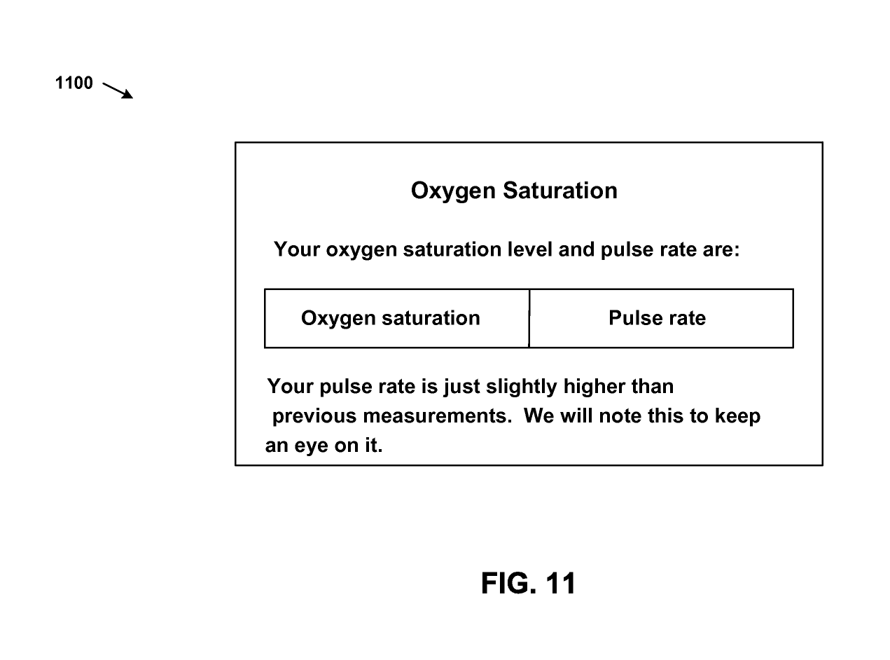

FIG. 11 shows an example result screen 1100 for oxygen saturation. The example result screen 1100 indicates the oxygen saturation reading, a pulse rate reading and a message. The message provides feedback to the patient regarding the oxygen saturation and pulse rate readings.

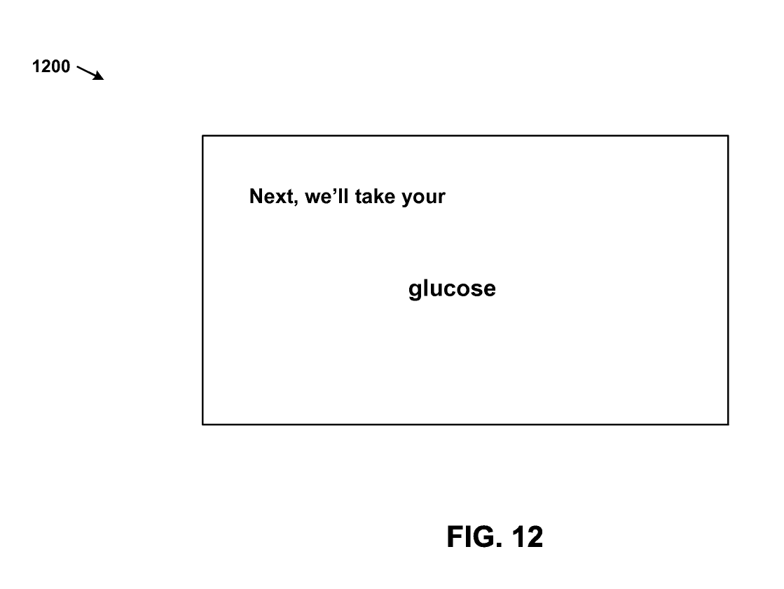

FIG. 12 shows an example screen 1200 that may be displayed after the result screen 1100. The screen 1200 indicates to the patient that the patient's glucose level is to be measured next.

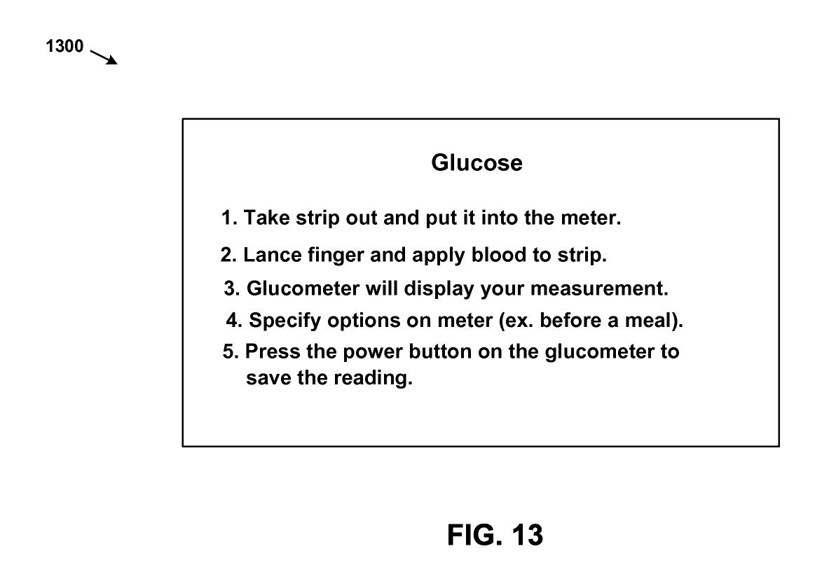

FIG. 13 shows an example instruction screen 1300 for measuring the patient's glucose level. The example instructions include 1) take a strip out and put it into the meter; 2) lance finger and apply blood to the strip; 3) the glucometer will display your measurement; 4) specify options on the meter (for example indicating that the glucose measurement was taken before a meal); and 5) press the power button on the glucometer to save the reading.

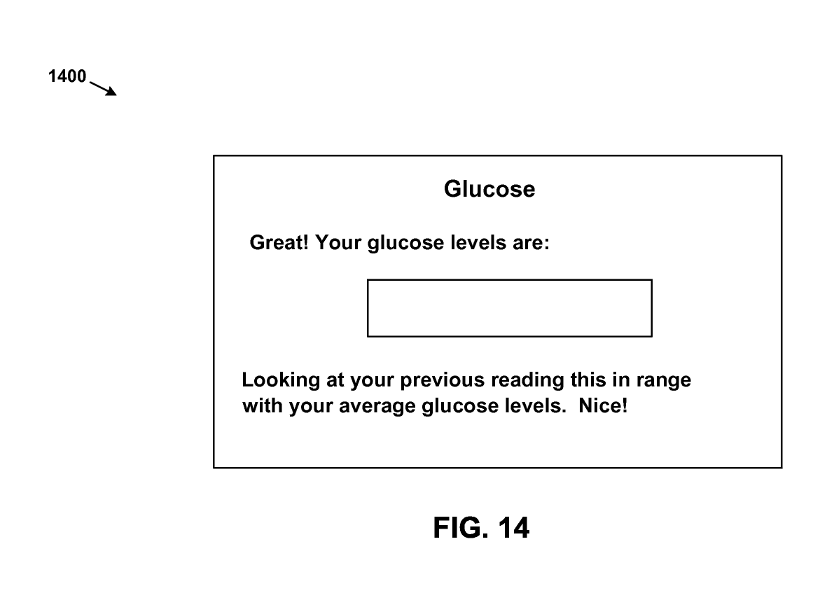

FIG. 14 shows an example result screen 1400 for glucose. The example result screen 1400 displays the glucose reading and a message. The message provides feedback to the patient regarding the glucose reading.

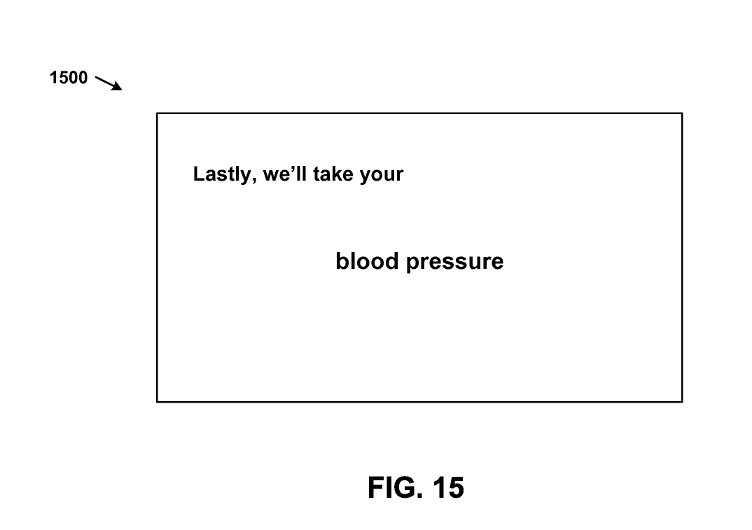

FIG. 15 shows an example screen 1500 that may be displayed after the result screen 1400. The screen 1500 indicates to the patient that the patient's blood pressure is to be measured next.

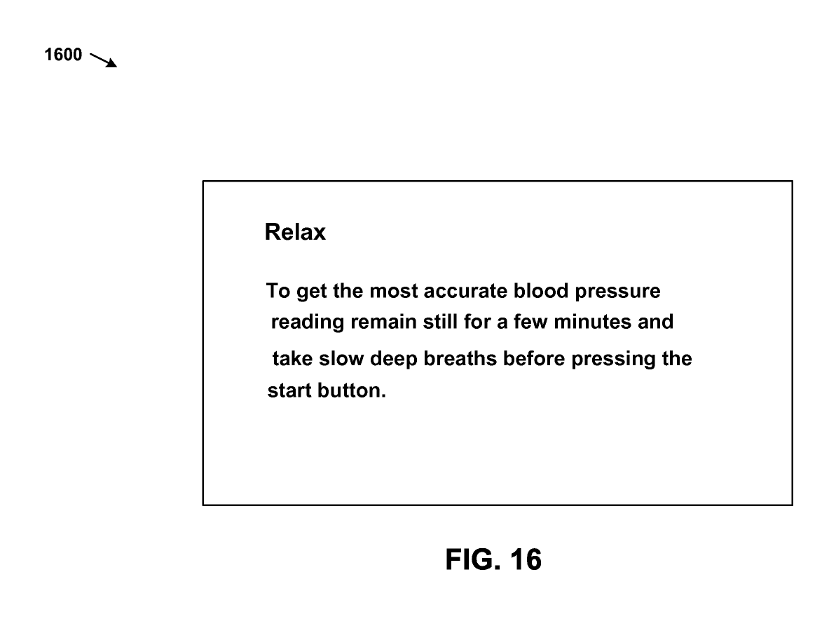

FIG. 16 shown an example instruction screen 1600 for a blood pressure measurement. The example instruction screen 1600 indicates that the patient should relax, remain still for a few minutes and take show breaths before pressing the start button.

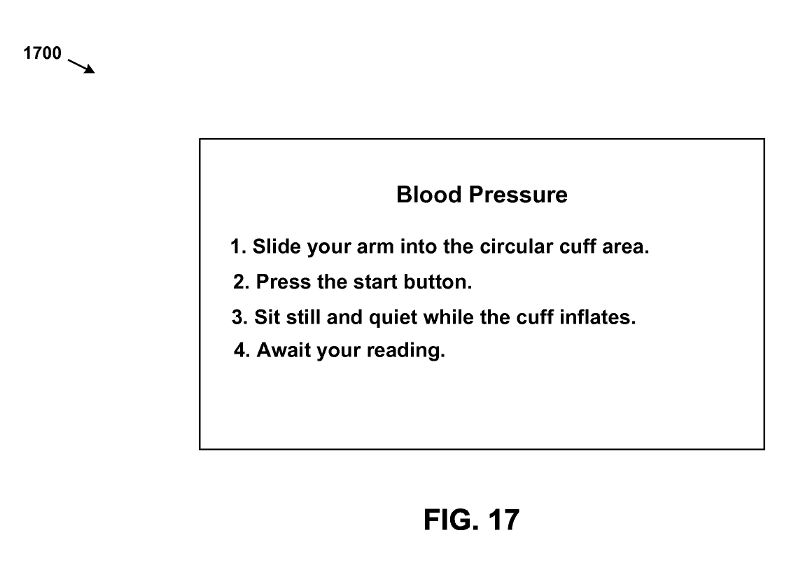

FIG. 17 show another example instruction screen 1700 for a blood pressure measurement. The example instructions include 1) slide your arm into the circular cuff area; 2) press the start button; 3) sit still and quiet while the cuff inflates; and 4) await your reading. More, fewer or different instruction screens may be displayed for a blood pressure measurement.

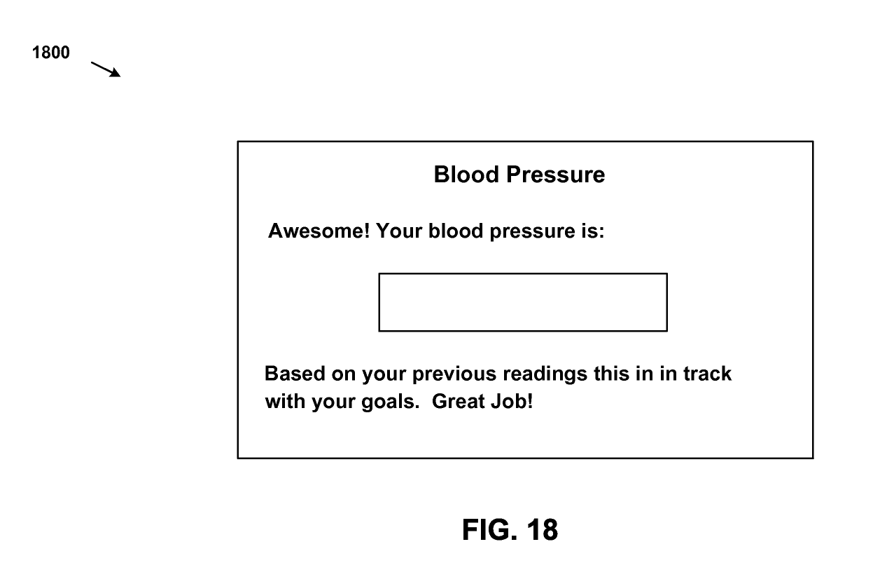

FIG. 18 shows an example result screen 1800 for blood pressure. The example result screen 1800 displays the blood pressure reading and a message. The message provides feedback to the patient regarding the blood pressure reading.

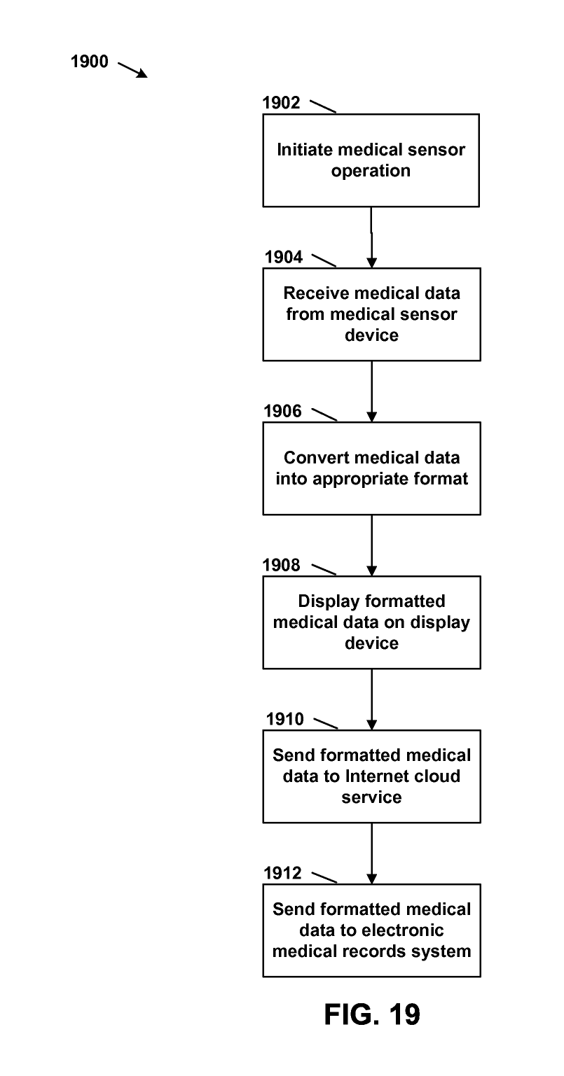

FIG. 19 shows an example flowchart for a method for obtaining medical data from a sensor using a remote health care medical device. The remote health care medical device is part of a remote health care system, for example system 100, that comprises patient 102, remote health care medical device 104, Internet cloud services 106 and EMR system 108.

At operation 1902, a medical sensor device operation is initiated. The medical sensor device operation may be one of obtaining a patient's blood pressure, measuring the patient's temperature, obtaining a glucose level for the patient, measuring the oxygen saturation level for the patient or some obtaining other medical data for the patient. The operation is initiated at the remote health care medical device 104. In some examples, the remote health care medical device 104 is programmed to initiate the medical sensor device operation at a particular day and time. In other examples, the patient may manually initiate the medical sensor device operation, for example by pressing a button on the remote health care medical device 104.

At operation 1904, medical data is received from the medical sensor device. The medical sensor data received from the medical sensor device is typically raw sensor data. Raw sensor data may include pressure data, heat data, blood data, electrical data, wavelength data, etc.

At operation 1906, the raw sensor data is converted into an appropriate format. Typically, each sensor device includes processing electronics that converts the raw data into the appropriate format. The processing electronics may include a processor, whereby a software algorithm running on the processor may implement the conversion. For example, pressure values from a blood pressure monitor may be converted into a systolic and diastolic blood pressure for the patient, heat data from an electronic thermometer may be converted into a temperature reading for the patient, blood data from a glucometer may be converted into a glucose level for the patient and wavelength data from an oxygen saturation sensor may be converted into an oxygen saturation reading for the patient.

At operation 1908, the formatted medical data is displayed on a display device. The display device is typically a display device that is part of the remote health care medical device 104, such as tablet computer 210 or a smart telephone. In some cases, for example for the remote health care medical device 104 shown in FIG. 2, the tablet computer 210 is embedded in the console of the remote health care medical device 104. In other examples, the tablet computer or smart telephone is externally secured to a base assembly of the remote health care medical device 104 via a mounting bracket.

At operation 1910, the formatted medical data is sent to an Internet cloud service. The Internet cloud service is typically a third party service, for example Amazon web services. In some examples, the Internet cloud service hosts a web site for the formatted medical data. The web site may be configured to obtain the formatted medical data from remote health care medical device 104 and to send the formatted medical data to a different destination, for example an EMR system.

At operation 1912, the Internet cloud service sends the formatted medical data to the EMR system, for example to EMR system 108. The EMR system stores the formatted medical data in a database on the EMR system or in an external database. In some examples, the third party cloud service is configured to send the formatted medical data to the EMR system when the formatted medical data is received at the third party cloud service. In other examples, the EMR system periodically polls the third party cloud service to request formatted medical data that is received at the third party cloud service. In some examples, the formatted medical data may be sent directly to a physician in addition to or in lieu of being sent to the EMR system.

Oral and visual prompts are used throughout the example method 1900 described above. In some examples the visual prompts shown in FIGS. 5-18 are used. In other examples, other visual prompts are used. Oral prompts may accompany one or more of the visual prompts. The visual and oral prompts aid the patient and make the remote health care medical device 104 easier to use.

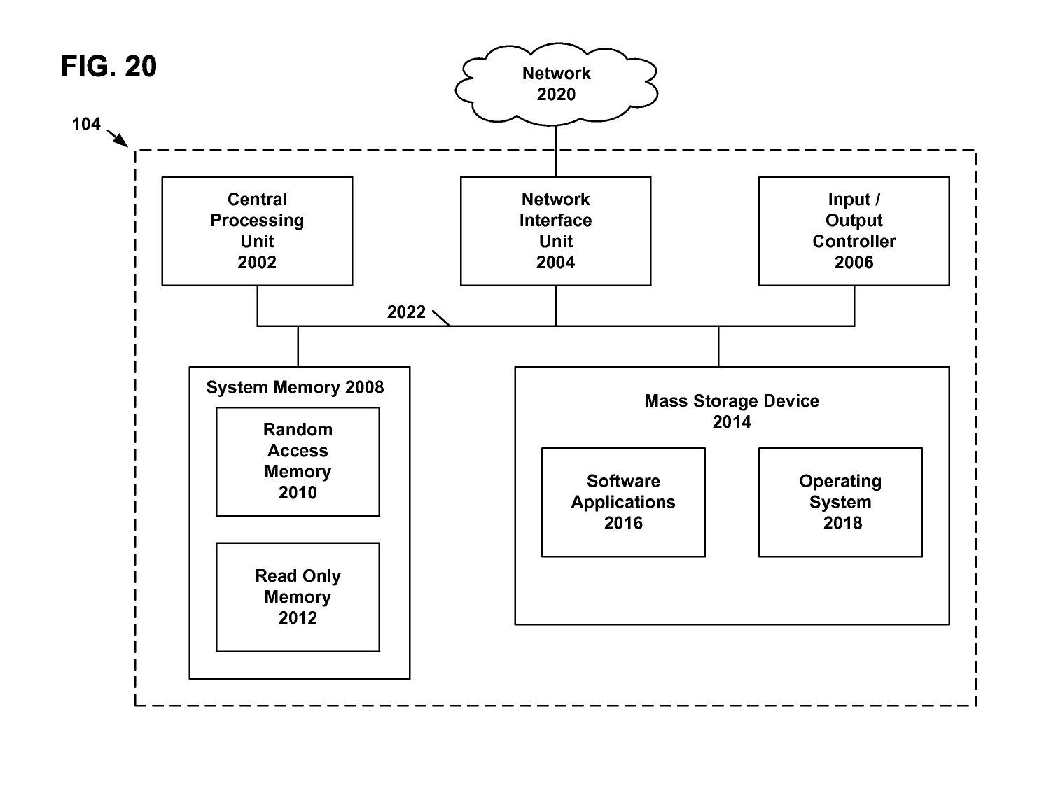

FIG. 20 illustrates example physical components of the remote health care medical device 104. As illustrated in the example of FIG. 20, the remote health care medical device 104 includes at least one central processing unit (“CPU”) 2002, a system memory 2008, and a system bus 2022 that couples the system memory 2008 to the CPU 2002. The system memory 2008 includes a random access memory (“RAM”) 2010 and a read-only memory (“ROM”) 2012. A basic input/output system contains the basic routines that help to transfer information between elements within the remote health care medical device 104, such as during startup, is stored in the ROM 2012. The remote health care medical device 104 further includes a mass storage device 2014. The mass storage device 2014 is able to store software instructions and data.

The mass storage device 2014 is connected to the CPU 2002 through a mass storage controller (not shown) connected to the bus 2022. The mass storage device 2014 and its associated computer-readable data storage media provide non-volatile, non-transitory storage for the remote health care medical device 104. Although the description of computer-readable data storage media contained herein refers to a mass storage device, such as a hard disk or solid state disk, it should be appreciated by those skilled in the art that computer-readable data storage media can be any available non-transitory, physical device or article of manufacture from which the central display station can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the remote health care medical device 104.

According to various embodiments of the invention, the remote health care medical device 104 may operate in a networked environment using logical connections to remote network devices through the network 2020, such as a local network, the Internet, or another type of network. The remote health care medical device 104 may connect to the network 2020 through a network interface unit 2004 connected to the bus 2022. It should be appreciated that the network interface unit 2004 may also be utilized to connect to other types of networks and remote computing systems. The remote health care medical device 104 also includes an input/output controller 2006 for receiving and processing input from a number of other devices, including a keyboard, a mouse, a touch user interface display screen, or another type of input device. Similarly, the input/output controller 2006 may provide output to a touch user interface display screen, a printer, or other type of output device.

As mentioned briefly above, the mass storage device 2014 and the RAM 2010 of the remote health care medical device 104 can store software instructions and data. The software instructions include an operating system 2018 suitable for controlling the operation of the remote health care medical device 104. The mass storage device 2014 and/or the RAM 2010 also store software instructions, that when executed by the CPU 2002, cause the remote health care medical device 104 to provide the functionality of the remote health care medical device 104 discussed in this document. For example, the mass storage device 2014 and/or the RAM 2010 can store software instructions that, when executed by the CPU 2002, cause the remote health care medical device 104 to display received physiological data on the display screen of the remote health care medical device 104.

Other example physical components of the remote health care medical device 104 are possible. In general, the remote health care medical device 104 supports a computing platform that provides capabilities for a standardized interface (for example USB) to retrieve data from specialized sensors, a standardized network communication interface (for example WiFi), a standardarized wireless communication interface (for example Bluetooth or NFC) and a user interface.

Although various embodiments are described herein, those of ordinary skill in the art will understand that many modifications may be made thereto within the scope of the present disclosure. Accordingly, it is not intended that the scope of the disclosure in any way be limited by the examples provided.

What is claimed is:

1. A health care medical device, the health care medical device comprising:
one or more medical sensor devices;
an assembly that houses the one or more medical sensor devices; and
a mobile electronic computing device with a display,
wherein the mobile electronic computing device is secured to the assembly, and wherein medical data from the one or more medical sensor devices is transmitted to the mobile electronic computing device.

2. The health care medical device of claim 1, wherein the mobile electronic computing device is enclosed within the assembly.

3. The health care medical device of claim 1, wherein the mobile electronic computing device is secured to an external section of the assembly.

4. The health care medical device of claim 1, wherein the mobile electronic computing device is a tablet computer.

5. The health care medical device of claim 1, wherein the mobile electronic computing device is a smart telephone.

6. The health care medical device of claim 1, wherein the medical data from the one or more medical sensor device is transmitted wirelessly to the mobile electronic computing device.

7. The health care medical device of claim 1, wherein the medical sensor devices include one or more of a blood pressure sensor, oxygen saturation sensor, thermometer and glucose meter.

8. The health care medical device of claim 7, further comprising a blood pressure sleeve that includes a blood pressure cuff, the blood pressure cuff automatically adjusting in position around an arm of a patient during a blood pressure measurement for the patient.

9. The heath care medical device of claim 1, wherein the mobile electronic computing device transmits the medical data from one or more of the medical sensor devices to an Internet-based hosting service.

10. The health care medical device of claim 1, wherein the mobile electronic computing device provides prompts to a user during the operation of the health care medical device.

11. The health care medical device of claim 10, wherein the prompts include both oral prompts and visual prompts.

12. The health care medical device of claim 1, further comprising a medical sensor device that is wearable by a user and that detects whether the user has fallen.

13. The health care medical device of claim 1, further comprising a video camera for viewing a user of the health care medical device.

14. The health care medical device of claim 13, further comprising receiving and processing responses from a clinician based on the clinician responding to one or more views from the video camera.

15. The health care medical device of claim 1, further comprising a sensor device that measures a weight of a patient.

16. A method of monitoring medical parameters of a patient using a health care medical device, the method comprising:
placing one or more medical sensors from the health care medical device on the patient;
receiving data from the one or more medical sensors;
processing the received data into medical parameters associated with the one or more medical sensors;
sending medical data based on the medical parameters to a mobile electronic computing device that is part of the health care medical device;
displaying the medical data on a display screen of the mobile electronic computing device;
making a wireless connection from the mobile electronic computing device to an Internet-based storage system; and
sending the medical data to the Internet-based storage system.

17. The method of claim 16, further comprising prompting the patient to operate one of more of the medical sensors.

18. The method of claim 17, wherein prompting the patient comprises providing oral and visual prompts to the patient.

19. The method of claim 16, further comprising using a camera that is included in the health care medical device to observer a status of the patient.

20. The method of claim 16, wherein the mobile electronic computing device is either a tablet computer or a smart telephone.

21. The method of claim 16, wherein the medical sensors include one or more of a blood pressure sensor, oxygen saturation sensor, thermometer and glucose meter.

22. The method of claim 16, further comprising receiving data from a sensor worn by the patient that monitors whether the patient has fallen.

23. A health care medical device, the health care medical device comprising:
one or more medical sensor devices, the medical sensor devices including one or more of a blood pressure sensor, oxygen saturation sensor, thermometer and glucose meter;
a tablet computer; and
an assembly that houses the one or more medical sensor devices and the tablet computer, the assembly enclosing the back and at least a part of four sides of the tablet computer,
wherein the tablet computer provides visual and oral prompts to a user during operation of the health care medical device and wherein the tablet computer transmits medical data from the one or more medical sensor devices to an Internet-based storage system.

24. The health care medical device of claim 23, further comprising a medical sensor device that is wearable by a user and that detects whether the user has fallen.

25. The health care medical device of claim 23, further comprising a video camera for viewing a user of the health care medical device.
